(19) United States
(12) Patent Application Publication
(10) Pub. No.: US 2023/0395267 A1
(43) Pub. Date: Dec. 7, 2023

(54) Procedure and Prediction System to Determine the Probability of a Patient Suffering from Sepsis

(71) Applicant: TCC GmbH, Hamburg (DE)

(72) Inventors: Alexander Krannich, Nauen (DE); Christian Storm, Hamburg (DE); David Barg, Hamburg (DE)

(21) Appl. No.: 18/330,426

(22) Filed: Jun. 7, 2023

(30) Foreign Application Priority Data
Jun. 7, 2022 (DE) 102022114248.5

(51) Int. Cl. G16H 50/30

(52) U.S. Cl. CPC G16H 50/30

(57) ABSTRACT

A procedure to determine the probability of a patient suffering from sepsis includes the following: successively detecting respective values for at least four predefined health-specific parameters of the patient over a predetermined detection period, determining an input value for each health-specific parameter, wherein this input value is dependent on a value greater than or equal to the 0.45 quantile and less than or equal to the 0.55 quantile of the values detected over the predetermined detection period successively for the respective health-specific parameter, inputting the input values for the four predefined health-specific parameters into a regression model or artificial neural network, wherein, when the input values are input, the regression model or artificial neural network provides a probability of the patient suffering from sepsis after a predetermined duration. This makes it possible to improve the accuracy of the indication of the probability of a patient suffering from sepsis.

The invention relates to a procedure to determine the probability of a patient suffering from sepsis, comprising the following procedure steps: detecting respective values for at least four predefined health-specific parameters and inputting the values for the predefined health-specific parameters into a regression model or artificial neural network implemented on a computer, wherein, when the input values are input, the regression model or artificial neural network provides a probability of the patient suffering from sepsis after a predetermined duration.

Sepsis is defined as a life-threatening situation which occurs when the body's own defensive reactions to an infection damage its own tissues and organs. It is one of the most serious complications of infectious diseases, which are triggered by bacteria, viruses, fungi, or parasites. For determining the probability of a patient suffering from sepsis, it is known to measure health-specific parameters and to pass them to a regression model or artificial neural network. The regression model or artificial neural network then uses these values to determine the probability of a patient suffering from sepsis for the patient. For instance, WO 2017/165693 A1 describes a disease prediction system comprising a processing circuit configured such that it receives a data set which contains data on a patient population, wherein the data contain, for each patient in a large number of patients in the patient population, values for a large number of features and a diagnostic value that indicates whether a disease has been diagnosed. The processing circuit is configured such that, on the basis of correlations between the values from the data set, it selects a large number of subsets of the features and, for each of at least one of the subsets, performs a machine-learning process using the respective subset and the diagnostic values as input parameters, wherein performing this process generates a respective prediction model. The processing circuit is configured such that it outputs the respective prediction model.

DE 10 2020 214 050 A1 describes a computer-implemented procedure for providing at least one predicted value for at least one medical laboratory variable, in particular for use in a medical laboratory value analysis, comprising the following steps: —providing at least one laboratory value progression which specifies a progression of historical laboratory values of the at least one laboratory variable at at least two historical points in time; —determining at least one laboratory variable feature for each of the at least one laboratory variable from the corresponding laboratory value progression; —determining the at least one predicted value at a predetermined prediction time on the basis of a trained, data-based prediction model and on the basis of the at least one laboratory variable feature for each of the at least one laboratory value progression.

WO 2006/061 644 A1 describes a system and a method for recognizing early signs of an infection and in particular for identifying persons in which sepsis is most likely. The measurement of the level of expression of certain combinations of cytokines and/or cellular activation markers, where necessary in combination with the use of prediction algorithms, allows for a high level of prediction accuracy. The method is applicable in both civilian and military sectors.

In this case, however, systems known in practice have thus far only provided low prediction reliability.

Proceeding therefrom, the problem addressed by the invention is to improve the accuracy of the indication of the probability of a patient suffering from sepsis.

This problem is solved by the subject matter of the independent claims. Preferred developments are found in the dependent claims.

According to the invention, a procedure is thus provided to determine the probability of the patient suffering from sepsis comprising the following procedure steps: successively detecting respective values for at least four predefined health-specific parameters of the patient over a predetermined detection period, determining an input value for each health-specific parameter, wherein this input value is dependent on a value greater than or equal to the 0.45 quantile and less than or equal to the 0.55 quantile of the values detected over the predetermined detection period successively for the respective health-specific parameter, inputting the input values for the predefined health-specific parameters into a regression model or artificial neural network implemented on a computer, wherein, when the input values are input, the regression model or artificial neural network provides a probability of the patient suffering from sepsis after a predetermined duration.

Health-specific parameters are all the measurable variables which can represent the state of health of a patient. They can be vital parameters and/or laboratory values or other measurable values that can be used for diagnostics. The predetermined detection period is the period in which the values of the health-specific parameters are used for determining the input values. The predetermined duration preferably covers a period of ten hours. This means that the probability of a patient suffering from sepsis is calculated over the following ten hours, such that, for example, a 72% probability of a patient suffering from sepsis prevails for the patient in question within the ten hours following the determining point.

A quantile is a measure of central tendency in statistics. The 0.45 quantile is the value at which 45% of all the values are less than or equal to this value. Determining an input value for each health-specific parameter, wherein this input value is dependent on a value greater than or equal to the 0.45 quantile and less than or equal to the 0.55 quantile of the values detected over the predetermined period successively for the respective health-specific parameter, means that a value from the range around the median of the values or the median itself is used to determine the input value. In the simplest form, the input value can be the median itself, but it can also be a value that deviates from the median. In addition, it does not have to be exactly the median or a value deviating from the median. Instead, it has been found in the context of the invention that those values originating from the midrange of the detected values sorted by size, namely those between the 0.45 quantile and the 0.55 quantile, can also be used as input values. Here too, the input value can be a value that deviates between these two quantiles. It is conceivable for a measuring instrument to be malfunctioning or not to be measuring at the intended position. This results in incorrect values or zero values within the predetermined detection period which, when these parameters are input into the model, give a NaN (“not a number”) response and would thus complicate the prediction. Even in the case of intermittent measurements or intermittent, defective measurement results, an accurate prediction can still be carried out according to the invention.

In principle, sepsis can be categorized on the basis of various health-specific parameters. According to a preferred development of the invention, it is, however, provided that the probability of a patient suffering from sepsis is determined with a qSOFA score of ≥2. The qSOFA score (quick single organ failure assessment score) is a reduced form of the SOFA score, which makes it possible to rapidly and advantageously assess a patient for an infection, with a qSOFA of ≥2 being deemed sepsis.

According to a preferred development of the invention, it is, however, provided that the predetermined detection period is between one hour and three hours. Preferably, the predetermined period is between 90 minutes and 150 minutes, more particularly preferably between 110 and 130 minutes. This predetermined period in which the health-specific parameters are acquired ensures that data is acquired both as early as possible and in a sufficient quantity for the probability of a patient suffering from sepsis to be determined as accurately as possible.

In principle, various combinations of health-specific parameters are possible for inputting into the regression model or artificial neural network. According to a preferred development of the invention, it is, however, provided that the health-specific parameters are determined using non-invasive measurement methods. As a result, it is possible to determine the probability of a patient suffering from sepsis using means that are easily accessible and rapidly available and do not have any tissue-damaging consequences. Non-invasive measurement methods are readily and routinely used in daily clinical practice in all areas of inpatient care, but also in particular in emergency care and intensive care and also in outpatient care in order to record health-specific parameters.

In principle, various combinations of health-specific parameters are possible for inputting into the regression model or artificial neural network. According to a preferred development of the invention, it is, however, provided that the health-specific parameters are oxygen saturation, heart rate, respiratory rate, and systolic blood pressure of the patient. These health-specific parameters can be obtained in full by routine patient monitoring and are additionally non-invasive. Likewise, these values can be retrieved at any point in time, provided that a corresponding measuring instrument is active. In time-critical assessments, such as determining the probability of a patient suffering from sepsis, this results in advantages over health-specific parameters that potentially require an imaging procedure or laboratory investigation.

It is possible that the probability of a patient suffering from sepsis is determined at a specified point in time. According to a preferred development of the invention, it is, however, provided that the probability of a patient suffering from sepsis is determined continuously. This can ensure that the patient is monitored in an uninterrupted manner, meaning that a flexible response can be given to rapidly changing health situations.

In principle, the probability of a patient suffering from sepsis can be determined in various ways. According to a preferred development of the invention, it is, however, provided that the procedure operates using an artificial neural network, which is trained to recognize sepsis with a qSOFA score of at least 2 in humans. Accordingly trained artificial neural networks provide reliable results for in particular non-linear, sufficiently complex problems. The correlations between health-specific parameters can therefore be very effectively modeled using artificial neural networks.

In principle, the input parameters can be assigned to the neurons in any combination. According to a preferred development of the invention, it is, however, provided that the artificial neural network comprises at least four neurons in an input layer, which are each assigned to one of the health-specific parameters. This means that correlations and influences of individual health-specific parameters can be taken into account in an improved manner and can be more clearly distinguished.

It is possible to configure the artificial neural network in various ways. According to a preferred development of the invention, it is, however, provided that the trained artificial neural network is configured with resilient propagation and with weighted tracking. Resilient propagation is an iterative procedure for determining the minimum of the error function in a neural network. Each neuron is connected to the following neuron in a further layer by an edge via a weight. The weights between the neurons are adjusted by the iterative process for minimizing the error function. The weighted tracking provides the adjustment of some or all the weights by means of heuristics. Resilient propagation algorithms are extremely robust in relation to their internal parameters, meaning that, together with the weighted tracking, an extremely robust network can be trained that has low susceptibility to noise.

In principle, various activation functions can be used in an artificial neural network. According to a preferred development of the invention, it is, however, provided that a single neuron is provided with one of the standard functions, such as logistic regression or the rectifier function, as the activation function. In artificial neural networks, each neuron forms a weighted total of its inputs and controls the resulting scalar value by means of a function, which is referred to as an activation function or transfer function. If the function is considered to be a linear function, the neuron carries out a linear regression or classification. In general, a non-linear function is used to carry out non-linear regressions and to solve classification problems that are not linearly separable. If the function is a sigmoid function that varies from 0 to 1 or −1 to 1, the output value of the neuron can be interpreted as a YES/NO response or a binary decision by means of a separation point.

In principle, the artificial neural network can be equipped with a large number of hidden layers. According to a preferred development of the invention, it is, however, provided that the artificial neural network is configured with exactly one input layer, exactly one hidden layer, and exactly one output layer. Neurons are combined into layers. Different layers can perform different transformations at their inputs. The signals migrate from the first layer (the input layer) to the last layer (the output layer).

It is possible for there to be a number of neurons in the hidden layer that is different from the input layer. According to a preferred development of the invention, it is, however, provided that the number of neurons in the hidden layer corresponds to the number of neurons in the input layer.

In principle, the artificial neural network can be provided with a different number of bias neurons. According to a preferred development of the invention, it is, however, provided that the artificial neural network is configured with two bias neurons. The bias neuron does not have an additional input within the neural network and always has a constant value, meaning that it can shift the results in the artificial neural network in a predefined direction as a neutral instance in combination with the weight.

According to the invention, a prediction system is provided for calculating the probability of the patient suffering from sepsis, wherein the prediction system comprises a database, an analysis unit, and a computer, wherein the successively detected values for the four predefined health-specific parameters of the patient over the predetermined detection period can be stored in the database and the input value can be determined by means of the analysis unit for each health-specific parameter that is greater than or equal to the 0.45 quantile and less than or equal to the 0.55 quantile of the values detected over the predetermined period successively for the respective health-specific parameter, wherein the input values for the predefined health-specific parameters can be input into the regression model or artificial neural network implemented on a computer, wherein, when the input values are input, the regression model or artificial neural network implemented on the computer can provide a probability of the patient suffering from sepsis after a predetermined duration.

The database preferably comprises an interface to a software system containing patient data and draws said data therefrom. In this case, the data can be drawn from systems such as a hospital information system (HIS), a patient data management system (PDMS), a system for outpatient documentation, or a software system of a mobile portable medical device or lifestyle product and are stored in a special structure such that they can be used by the analysis unit. The database can function as the software framework, which makes it possible to display, provide, manage, and control access to the data set. The database can make it possible to compile, read, update, and delete required data in the data set. It can function as an interface between the programs and data. To do this, the data in the database are consistent and accurate. This means that the data in all the databases are consistent and correct for all users. The database management system provides access to well-managed and synchronized data. In addition, data consistency in the database is ensured; there is no data redundancy. Furthermore, any modifications to the database are preferably reflected immediately. The database management system helps to give rapid responses to queries, meaning that the data is accessed accurately and more rapidly. The analysis unit can be configured in the computer which contains the regression model or the artificial neural network.

In principle, the health-specific parameters can be transmitted at specified points in time. According to a preferred development of the invention, it is, however, provided that the health-specific parameters of the patients are linked to the medical documentation system in the database via an interface, wherein the health-specific parameters can be transmitted continuously. This makes it possible to continuously calculate the probability of the patient suffering from sepsis after a predetermined duration.

According to a preferred development of the invention, it is provided that the prediction system comprises a display unit and the predetermined detection period for the patient can be controlled via the display unit provided with a user interface. This makes it possible to give a flexible response to the measured parameters and to the probability of the patient suffering from sepsis after a predetermined duration. When the risk is low, resources can potentially be conserved by way of shorter predetermined detection periods. When the risk is high, however, more accurate monitoring can be achieved by way of longer predetermined detection periods. The display unit can be configured in the computer which contains the regression model or the artificial neural network.

According to a preferred development of the invention, it is provided that the probability of the patient suffering from sepsis after a predetermined duration is continuously visible in the user interface.

It is possible that only the current probability of the patient suffering from sepsis after a predetermined duration can be retrieved. According to a preferred development of the invention, it is, however, provided that the probability of the patient suffering from sepsis after a predetermined duration can be retrieved as a progression over time for the patient in question.

In the drawings:

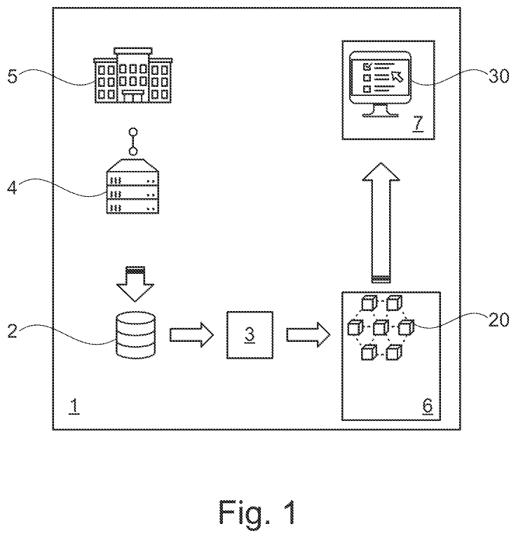

FIG. 1 schematically shows a prediction system for calculating the probability of a patient suffering from sepsis according to a preferred exemplary embodiment of the invention,

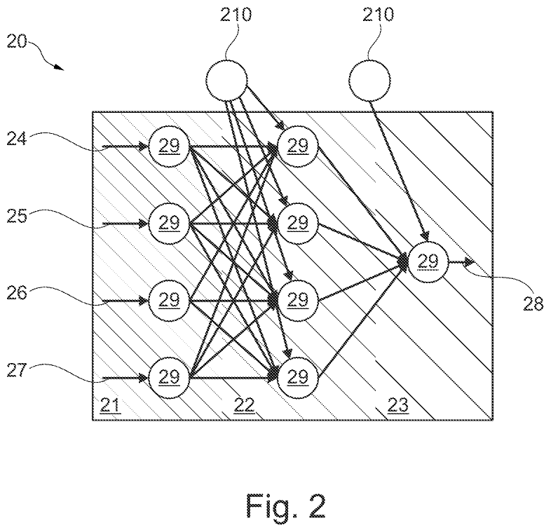

FIG. 2 schematically shows an artificial neural network according to a preferred exemplary embodiment of the invention, and

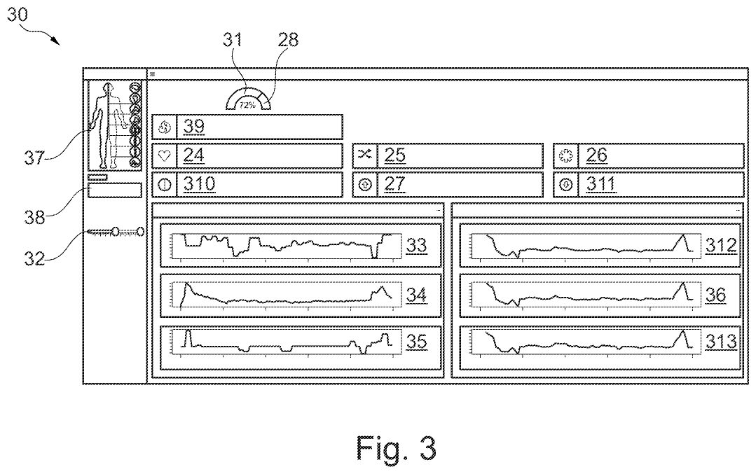

FIG. 3 schematically shows a user interface according to a preferred exemplary embodiment of the invention.

FIG. 1 schematically shows a prediction system 1 to determine the probability of a patient 37 suffering from sepsis, comprising a database 2, an analysis unit 3, and a computer 6. A patient 37 is on an intensive care unit of a hospital 5 and is connected to a ventilatory support device, a blood pressure cuff, and a pulse oximeter, thus meaning that four different non-invasive health-specific parameters can be obtained as values: heart rate 33, respiratory rate 34, oxygen saturation 35, and systolic blood pressure 36. These health-specific parameters are measured continuously and the values are stored in an automated manner in a hospital system 4, which is linked to a database 2 via an interface.

The database 2 draws the measured values continuously and formats them such that the input value for each health-specific parameter can be determined by the analysis unit 3. This unit likewise continuously accesses the health-specific parameters contained in the database 2. For a predetermined detection period 32, which can be controlled variably in the user interface 30 of the display unit 7, all the values for each health-specific parameter for the patient are loaded into the analysis unit 3. The input values for the computer 6 and the artificial neural network 20 contained therein are determined for each health-specific parameter by means of the analysis unit 3, wherein this input value is dependent on a value greater than or equal to the 0.45 quantile and less than or equal to the 0.55 quantile of the values detected over the predetermined detection period 32 successively for the respective health-specific parameter.

The probability 28 of the patient suffering from sepsis after a predetermined duration, as determined by the artificial neural network 20 in the computer 6, is provided by means of the user interface 30 reproduced by the display unit 7.

The artificial neural network 20 shown in FIG. 2 comprises an input layer 21, a hidden layer 22, and an output layer 23. The input values determined by the analysis unit 3 for the heart rate 24, respiratory rate 25, oxygen saturation 26, and systolic blood pressure 27 are passed to the artificial neural network 20 within the input layer 21. Each neuron 29 transfers an input, for example p features (x₁ᵢ, . . . , xₚᵢ) which describe a patient i, into an output y(x), where

$$y ( x ) = f ( {β}_{0} + {\sum }_{l = 1}^{p} {β}_{l} {x}_{l} ) .$$

The probability of belonging to a class is determined by the incoming data being multiplied by certain weights βₗ, l=0, 1, . . . , p and the activation function ƒ being applied and added up.

Each neuron 29 in the input layer 21 is connected to each neuron 29 in the hidden layer 22 by an edge via a weight. The number of neurons 29 in the hidden layer 22 of the artificial neural network 20 in FIG. 2 matches the number of neurons 29 in the input layer 21. The output layer 23 comprises a single neuron 29. Each neuron 29 in the hidden layer 22 is connected to the single neuron 29 in the output layer 23 by an edge via a weight. In addition, two bias neurons 210 are contained in the artificial neural network 20. The first bias neuron 210 is located between the input layer 21 and the hidden layer 22 and is connected to each neuron 29 in the hidden layer 22 by an edge via a weight. The second bias neuron 210 is located between the hidden layer 22 and the output layer 23 and is connected to the neuron 29 in the output layer 23 by an edge via a weight. The neuron 29 in the output layer 23 provides the probability 28 of the patient suffering from sepsis after a predetermined duration. For this purpose, the sepsis is classified by a qSOFA score of at least 2.

The performance of the artificial neural network 20 is determined by a diagnostic test. It is used to assess the diagnostic accuracy or prediction accuracy. The finding in relation to sepsis can be positive or negative. It should be added that this finding is not always correct. Therefore, a distinction must also be drawn between true positive (TP), true negative (TN), false positive (FP), and false negative (FN). The associated frequencies are shown in the following table.

TABLE 1
Fourfold table for the diagnostic test.

| | Test | | |
|---|---|---|---|
| Finding | positive | negative | Total |
| present | a = TP | b = FN | a + b |
| | | | [Persons affected] |
| absent | c = FP | d = TN | c + d |
| | | | [Persons not |
| | | | affected] |
| Total | a + c | b + d | a + b + c + d = n |
| | [Test positive] | [Test negative] | |

More generally and similarly to table 1, this can be represented by the corresponding mathematical relationships:

TABLE 2
General fourfold table for the diagnostic test.

| | T⁺ | T⁻ | Σ |
|---|---|---|---|
| K | a | b | a + b |
| K̅ | c | d | c + d |
| Σ | a + c | b + d | n |

In this case, important characteristic variables for the diagnostic test are
1. the proportion of persons testing negative of the persons not affected,
2. the proportion of persons testing positive of the persons affected,
3. the proportion of persons not affected of the persons testing negative, and
4. the proportion of persons affected of the persons testing positive.

These four variables are denoted by:
1. Specificity:

$$P ( {T}^{-} \vert \overline{K} ) = \frac{a}{a + b} ,$$

2. Sensitivity:

$$P ( {T}^{+} \vert K ) = \frac{d}{c + d} ,$$

3. Negative predictive value (NPV):

$$P ( \overline{K} \vert {T}^{-} ) = \frac{d}{b + d}$$

and
4. Positive predictive value (PPV):

$$P ( K \vert {T}^{+} ) = \frac{a}{a + c} .$$

Other important variables for the diagnostic test are the prevalence and the probability quotients. The prevalence gives the proportion of ill persons in the sample. The positive probability quotient gives the probability of finding a positive test result in the persons affected compared with the probability of finding a positive test result in the persons not affected (LR⁺). Similarly, LR⁻ can be interpreted for a negative test result:

$${LR}^{+} = \frac{Sensitivity}{1 - Specificity} , {LR}^{+} = \frac{1 - Sensitivity}{Specificity} .$$

LR⁺ and LR⁻ have the advantage that they are independent of the prevalence and factor in Sensitivity and Specificity.

Since the measured variable for predicting the finding is metric, it is necessary to determine a limit value. This limit value is obtained by Sensitivity and 1−Specificity being plotted against one another on a graph. The ideal limit value results from a parallel shift of the bisecting line at the outermost point of contact of the curve. At this point, the ideal limit value is c, for which the total of Sensitivity and Specificity is at a maximum. This maximum total is referred to as the Youden index, where

J_{c}=max(Sensitivity(c)+Specificity(c)−1)

The performance for providing the probability 28 of the patient suffering from sepsis after a predetermined duration is shown in table 3.

TABLE 3
Prediction accuracy.

| Index | Value |
|---|---|
| PPV | 0.69 |
| NPV | 0.839 |
| Sensitivity | 0.853 |
| Specificity | 0.667 |
| Limit value | −0.461 |
| RP | 29 |
| RN | 26 |
| FP | 13 |
| FN | 5 |
| AUC (95%- | 0.814(0.717 − |
| CI) | 0.912) |
| LR+ | 2.559 |
| LR− | 0.221 |
| Youden | 0.52 |
| index | |

FIG. 3 shows the user interface 30, which displays the input values of the health-specific parameters: heart rate 24, respiratory rate 25, oxygen saturation 26, and systolic blood pressure 27. Furthermore, the continuously detected values of the health-specific parameters of heart rate 33, respiratory rate 34, oxygen saturation 35, and systolic blood pressure 36 are plotted against the time of the predetermined detection period 32 in the user interface. These data can be retrieved for any patient 37 for which they have been recorded via an input in a selection region 38 by the patient 37 in question being switched via the selection region 38. The probability 28 of the patient 37 suffering from sepsis after a predetermined duration is provided for any patient 37. This probability of the patient suffering from sepsis is visualized as a tachometer 31 and as an output 39. The output 39 additionally displays the predetermined detection period 32. Further input values of important parameters, such as a mean arterial pressure 310 and a diastolic pressure 311, as well as the associated values 312 and 313, can likewise be displayed.

### LIST OF REFERENCE SIGNS

1 Prediction system
2 Database
3 Analysis unit
4 Hospital system
5 Hospital
6 Computer
7 Display unit
20 Artificial neural network
21 Input layer
22 Hidden layer
23 Output layer
24 Heart rate input value
25 Respiratory rate input value
26 Oxygen saturation input value
27 Systolic blood pressure input value
28 Probability of the patient suffering from sepsis after a predetermined duration
29 Neuron
210 Bias neuron
30 User interface
31 Tachometer
32 Predetermined detection period
33 Heart rate values
34 Respiratory rate values
35 Oxygen saturation values
36 Systolic blood pressure values
37 Patient
38 Selection region
39 Output
310 Mean arterial pressure input value
311 Diastolic pressure input value
312 Mean arterial pressure values
313 Diastolic pressure values

1. A procedure to determine the probability of a patient suffering from sepsis, the procedure comprising:
successively detecting respective values for at least four predefined health-specific parameters of the patient over a predetermined detection period,
determining an input value for each health-specific parameter, wherein this input value is dependent on a value greater than or equal to the 0.45 quantile and less than or equal to the 0.55 quantile of the values detected over the predetermined detection period successively for the respective health-specific parameter,
inputting the input values for the predefined health-specific parameters into a regression model or artificial neural network implemented on a computer, wherein, when the input values are input, the regression model or artificial neural network provides a probability of the patient suffering from sepsis after a predetermined duration.

2. The procedure to determine the probability of a patient suffering from sepsis according to claim 1, wherein the probability of a patient suffering from sepsis is determined with a qSOFA score of ≥2.

3. The procedure to determine the probability of a patient suffering from sepsis according to claim 1, wherein the predetermined detection period is between one hour and three hours.

4. The procedure to determine the probability of a patient suffering from sepsis according to claim 1, wherein the health-specific parameters are determined using non-invasive measurement methods.

5. The procedure to determine the probability of a patient suffering from sepsis according to claim 1, wherein the health-specific parameters are oxygen saturation, heart rate, respiratory rate, and systolic blood pressure of the patient.

6. The procedure to determine the probability of a patient suffering from sepsis according to claim 1, wherein the probability of a patient suffering from sepsis is determined continuously.

7. The procedure to determine the probability of a patient suffering from sepsis according to claim 1, wherein the procedure operates using an artificial neural network, which is trained to recognize sepsis with a qSOFA score of at least 2 in humans.

8. The procedure to determine the probability of a patient suffering from sepsis according to claim 7, wherein the artificial neural network comprises at least four neurons in an input layer, which are each assigned to one of the health-specific parameters.

9. The procedure to determine the probability of a patient suffering from sepsis according to claim 7, wherein the trained artificial neural network is configured with resilient propagation and with weighted tracking.

10. The procedure to determine the probability of a patient suffering from sepsis according to claim 7, wherein the artificial neural network is configured with an input layer, a hidden layer, and an output layer.

11. The procedure to determine the probability of a patient suffering from sepsis according to claim 10, wherein the number of neurons in the hidden layer corresponds to the number of neurons in the input layer.

12. A prediction system for calculating the probability of a patient suffering from sepsis, wherein the prediction system comprises a database, an analysis unit, and a computer, wherein
successively detected values for at least four predefined health-specific parameters of a patient over a predetermined detection period can be stored in the database and
an input value can be determined by means of the analysis unit for each health-specific parameter that is greater than or equal to the 0.45 quantile and less than or equal to the 0.55 quantile of the values detected over a predetermined detection period successively for the respective health-specific parameter, wherein
the input values for the predefined health-specific parameters can be input into a regression model or artificial neural network implemented on a computer,
wherein, when the input values are input, the regression model or artificial neural network implemented on the computer can provide a probability of the patient suffering from sepsis after a predetermined duration.

13. The prediction system for calculating the probability of a patient suffering from sepsis according to claim 12, wherein the probability of the patient suffering from sepsis after a predetermined duration can be calculated using a computer provided with a GPU architecture.

14. The prediction system for calculating the probability of a patient (37) suffering from sepsis according to claim 12, wherein the health-specific parameters of the patients are linked to the hospital system in the database via an interface, wherein the health-specific parameters can be transmitted continuously.

15. The prediction system for calculating the probability of a patient suffering from sepsis according to claim 12, wherein the prediction system additionally comprises a display unit and the predetermined detection period for the patient can be controlled via the display unit provided with a user interface.
